(19) United States
(12) Patent Application Publication
(10) Pub. No.: US 2025/0263650 A1
(43) Pub. Date: Aug. 21, 2025

(54) FUNGUS AND MICROBIAL AGENT FOR TREATING MERCURY CONTAMINATION, USE THEREOF, MERCURY REMOVAL METHOD, AND METHOD FOR IDENTIFYING FUNGUS CAPABLE OF TREATING MERCURY CONTAMINATION

(71) Applicant: ZHEJIANG UNIVERSITY, Hangzhou, Zhejiang (CN)

(72) Inventors: Weiguo FANG, Hangzhou, Zhejiang (CN); Congcong WU, Hangzhou, Zhejiang (CN)

(21) Appl. No.: 17/775,404

(22) PCT Filed: Dec. 8, 2021
(86) PCT No.: PCT/CN2021/136429

§ 371 Date: May 9, 2022

(30) Foreign Application Priority Data
Nov. 24, 2021 (CN) 202111402942.9

(51) Int. Cl. C12N 1/14; B09C 1/10; C02F 3/34; C02F 101/20; C12N 9/02; C12N 9/88; C12R 1/645

(52) U.S. Cl. CPC C12N 1/14; B09C 1/105; C02F 3/347; C12N 9/0091; C12N 9/88; C02F 2101/20; C12R 2001/645; C12Y 116/01001; C12Y 499/01002

(57) ABSTRACT

The present disclosure provides a fungus and a microbial agent for treating mercury contamination, use thereof, a mercury removal method, and a method for identifying a fungus capable of treating mercury contamination, and relates to the technical field of biological mercury removal. The present disclosure provides a Metarhizium fungus and 8 species of non-Metarhizium fungi for treating mercury contamination, a method for identifying a fungus capable of treating mercury contamination, and enzymes for removing methylmercury and divalent mercury, which provides a genetic basis for treating mercury contamination with recombinant fungi or bacteria.

### CROSS REFERENCE TO RELATED APPLICATIONS

The present application is a national stage application of International Patent Application No. PCT/CN2021/136429, filed on Dec. 8, 2021, which claims priority to the Chinese Patent Application 202111402942.9, filed to the China National Intellectual Property Administration (CNIPA) on Nov. 24, 2021 and entitled “FUNGUS AND MICROBIAL AGENT FOR TREATING MERCURY CONTAMINATION, USE THEREOF, MERCURY REMOVAL METHOD, AND METHOD FOR IDENTIFYING FUNGUS CAPABLE OF TREATING MERCURY CONTAMINATION”, which is incorporated herein by reference in its entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the file created on Mar. 20, 2024, entitled “SEQUENCE LISTING.txt”, with 5,475 bytes in size.

### TECHNICAL FIELD

The present disclosure belongs to the technical field of biological mercury removal, and specifically relates to a fungus and a microbial agent for treating mercury contamination, use thereof, a mercury removal method, and a method for identifying a fungus capable of treating mercury contamination.

### BACKGROUND ART

Mercury is a natural component in the earth's crust and persists in the environment, which is currently known to be the only liquid heavy metal element under normal temperature and pressure. Mercury exists in the environment in the form of inorganic or organic mercury. Inorganic mercury mainly includes metallic mercury, mercurous ions (Hg₂²⁺), and divalent mercury ions (Hg²⁺). Divalent mercury ions can be covalently bonded with a carbon atom to form an organic mercury compound, such as methylmercury (MeHg).

Divalent mercury ions (Hg²⁺) have high electron affinity, and can be covalently bonded with an electron donor (such as sulfur, oxygen, and nitrogen)-containing group, such as sulfhydryl, carbonyl, carboxyl, hydroxyl, amino, and phosphoryl. These groups are the most important active groups in organisms, and will lose activity after being covalently bonded with Hg²⁺. Therefore, Hg²⁺ has a huge impact on the physiological and biochemical functions of organisms, and causes great harm to organisms including humans. Methylmercury also reacts with sulfhydryl of a protein, making the protein molecule undergo a mercuration reaction and lose activity. For humans, methylmercury is extremely neurotoxic. Methylmercury is easily absorbed by animals and plants to accumulate in the food chain and biosphere, thereby contaminating agricultural products, freshwater products, and seafood and seriously threatening the food safety, life, and health of humans.

Due to characteristics such as high toxicity, persistence, bioaccumulation, and long-distance transmission, mercury is considered to be one of the three most dangerous metal elements. Mercury has been listed as one of 129 hazardous chemicals by the Environmental Protection Agency (EPA) of the United States, and has also been included in the Carcinogen List of the International Agency for Research on Cancer (IARC) of the World Health Organization (WHO). At present, the mercury contamination is mostly treated by passivating agents or bacteria, and there is no report on the treatment of mercury contamination by fungi.

### SUMMARY

In view of this, the present disclosure is intended to provide a fungus and a microbial agent for treating mercury contamination, use thereof, a mercury removal method, and a method for identifying a fungus capable of treating mercury contamination. The use of the discovered wild-type fungus to treat mercury contamination results in no genetic contamination, and shows high efficiency for removing methylmercury and divalent mercury contamination in the environment.

To achieve the objective of the present disclosure, the present disclosure provides the following technical solutions.

The present disclosure provides a fungus for treating mercury contamination, where the fungus expresses a methylmercury demethylase (MMD) and a mercury ion reductase (MIR); and
the fungus includes a Metarhizium fungus and a non-Metarhizium fungus, and the non-Metarhizium fungus includes Fusarium oxysporum, Oidiodendron maius, Pyronema omphalodes, Amorphotheca resinae, Cadophora malorum, Hyaloscypha bicolor, Pseudogymnoascus sp, and Exophiala oligosperma.

Preferably, a gene encoding the MMD may have a Genbank accession number of XP_007825874; and a gene encoding the MIR may have a Genbank accession number of XP_007824121.

Preferably, the Metarhizium fungus may include Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii may have an accession number of USDA ARSEF2575, the M. anisopliae may have an accession number of USDA ARSEF549, the M. brunneum may have an accession number of USDA ARSEF3297, the M guizhouense may have an accession number of USDA ARSEF977, the M. majus may have an accession number of USDA ARSEF297, and the M. acridum may have an accession number of USDAARSEF324; and
the Fusarium oxysporum may have an accession number of NRRL 32931, the Cadophora malorum may have an accession number of bio-12245, the Oidiodendron maius may have an accession number of ATCC 60377, the Hyaloscypha bicolor may have an accession number of CBS144009, the Pseudogymnoascus sp. may have an accession number of ATCC MYA-4855, the Pyronema omphalodes may have an accession number of ATCC 14881, the Exophiala oligosperma may have an accession number of ATCC28180, and the Amorphotheca resinae may have an accession number of bio-104132.

The present disclosure also provides a microbial agent for methylmercury demethylation and divalent mercury reduction, including at least one of the fungi described above.

The present disclosure also provides use of the fungus or the microbial agent described above in the removal of mercury contamination.

The present disclosure also provides a filter element for methylmercury demethylation and divalent mercury reduction, where the filter element uses hyphae of at least one of the fungi described above as a filler.

The present disclosure also provides a filter device for removing methylmercury and divalent mercury in water, including the filter element.

The present disclosure also provides a method for removing methylmercury and divalent mercury in water, including the following steps: placing the microbial agent in the water and stirring for more than 48 h, or filtering the water through the filter element or the filter device.

The present disclosure also provides a method for removing methylmercury and divalent mercury in soil, including the following steps: planting a plant that has a symbiotic relationship with the fungus in the soil, and inoculating the fungus.

The present disclosure also provides a method for identifying a non-Metarhizium fungus capable of methylmercury demethylation, including the following step: identifying whether a genome of the non-Metarhizium fungus includes Metarhizium MMD and MIR coding genes or homologous genes of the MMD and MIR coding genes.

Beneficial effects: The present disclosure provides a fungus for treating mercury contamination, and specifically discovers a wild-type Metarhizium fungus and 8 species of non-Metarhizium fungi that can remove environmental methylmercury and divalent mercury contamination, and genes/proteins for removing methylmercury and divalent mercury (MMD and MIR). The discovery of the genes/proteins of the present disclosure provides a genetic basis for treating mercury contamination with recombinant bacteria.

When the fungus or microbial agent provided by the present disclosure is applied to water, heavy metal mercury in the water can be removed by a method such as cultivation or filtration. When the fungus or microbial agent provided by the present disclosure is applied to soil, a plant that can form a symbiotic relationship with the fungus is planted on contaminated soil, then the fungus is inoculated, and the fungus can grow in a rhizosphere of the plant using nutrients secreted by the rhizosphere to eliminate methylmercury and divalent mercury in the soil and reduce the accumulation of methylmercury and divalent mercury in the plant, thereby achieving the effect of treating mercury contamination.

In an embodiment of the present disclosure, when a methylmercury concentration in freshwater or seawater is 50 μg/l, the Metarhizium can completely remove methylmercury in the water. When the methylmercury concentration is increased to 1 mg/L, the Metarhizium can completely remove methylmercury in the freshwater or seawater. When freshwater with a methylmercury concentration of 5 mg/L is treated with the Metarhizium, 50% of the methylmercury is removed; and when seawater with a methylmercury concentration of 5 mg/L is treated with the Metarhizium, 70% of the methylmercury is removed. When the Metarhizium is used to treat divalent mercury, at a divalent mercury concentration of 0.5 mg/L, the Metarhizium can completely remove the divalent mercury in water; at a divalent mercury concentration increased to 1 mg/L, 70% of the divalent mercury can be removed; and in freshwater and seawater both with a divalent mercury concentration of 5 mg/L and a divalent mercury concentration of 10 mg/L, 50% of the divalent mercury can be removed by the Metarhizium.

After the Metarhizium is inoculated in the soil, the accumulation of methylmercury and divalent mercury in a plant is significantly reduced; and compared with an uninoculated plant, a methylmercury content in the plant inoculated with the Metarhizium is decreased by 2.58 times, where a methylmercury content in the aboveground part is decreased by 2 times and a methylmercury content in the underground part is decreased by 2.52 times, and similarly, a divalent mercury content in the plant is decreased by 4.19 times, where a divalent mercury content in the aboveground part is decreased by 3 times and a divalent mercury content in the underground part is decreased by 6.2 times. After the Metarhizium is inoculated, a methylmercury content in the rhizosphere soil of the plant is decreased by 1.2 times, and a divalent mercury content in the rhizosphere soil is decreased by 1.1 times.

### Biological Deposit Information

M. robertsii was deposited on Jul. 21, 1988, with an accession number of ARSEF2575; M. anisopliae was deposited in September 1980, with an accession number of ARSEF549; M. majus was deposited on Sep. 22, 1978, with an accession number of ARSEF297; M brunneum was deposited in 1987, with an accession number of ARSEF3297; M. acridum was deposited in February 1979, with an accession number of ARSEF324; and M guizhouense was deposited on Oct. 17, 1983, with an accession number of ARSEF977. The above 6 Metarhizium strains were all deposited in the Agricultural Research Service Collection of Entomopathogenic Fungal Cultures (ARSEF), which belongs to the Agricultural Research Service Culture Collection (NRRL) of the United States (1815 N. University Street Peoria, IL 61604).

Fusarium oxysporum was deposited in 1999 in the University of Texas Health Science Center at San Antonio, Texas, United States, with an accession number of NRRL 32931.

Cadophora malorum was deposited on Feb. 14, 2007, with an accession number of bio-12245. Cadophora malorum was originally from Centraalbureauvoor Schimmelcultures (CBS), with an original number of CBS 100591.

Amorphotheca resinae was deposited on May 19, 1947, with an accession number of Bio-104132. Amorphotheca resinae was from CBS, with an original number of CBS 186.54.

### BRIEF DESCRIPTION OF DRAWINGS

FIG. 1 shows a filter with hyphae as a matrix;

FIG. 2 shows the mercury removal capacity of the filter, where A shows a methylmercury content in a filtrate obtained after each of three times of filtration in fresh water, and B shows a Hg²⁺ content in a filtrate obtained after each of three times of filtration in fresh water; C shows a methylmercury content in a filtrate obtained after each of three times of filtration in sea water, and D shows a Hg²⁺ content in a filtrate obtained after each of three times of filtration in sea water;

FIG. 3 shows verification results of the construction of gene Mmd and Mir-knockout mutants and a complementary strain thereof, where A shows a verification result of the Mmd-knockout mutant and B shows a verification result of the Mir-knockout mutant, with a PCR amplification result using Bar-up and CF-2 as primers shown in the upper panel and a PCR amplification result using CF-1 and CF-2 as primers shown in the lower panel; C shows a verification result of the complementary strain, with a PCR amplification result using cc-Mmd-5 and cc-Mmd-3 as primers shown in the left panel and a PCR amplification result using cc-Mir-5 and cc-Mir-3 as primers shown in the right panel; D shows gene knockout based on the principle of homologous recombination, with a location of the target gene in the fungal genome shown in the upper panel and a gene knockout plasmid map shown in the lower panel; and E shows verification results of Mmd and Mir in a double-knockout mutant, with primers consistent with those in A and B;

FIG. 4 shows the growth of Zea mays L. plants inoculated with or without M. robertsii spores in soil with 10 μg/kg methylmercury or 20 mg/kg divalent mercury (aboveground seedling height), where A shows the growth of Zea mays L. plants inoculated with or without M. robertsii spores in soil with methylmercury; and B shows the growth of Zea mays L. plants inoculated with or without M. robertsii spores in soil with divalent mercury;

FIG. 5 shows determination results of dry weights and fresh weights of the aboveground part (seedling) and underground part (root) of a Zea mays L. plant in soil with methylmercury or divalent mercury, where A shows the dry weight and fresh weight of the aboveground part (seedling) of the Zea mays L. plant in soil with methylmercury; B shows the dry weight and fresh weight of the underground part (root) of the Zea mays L. plant in soil with methylmercury; C shows the dry weight and fresh weight of the aboveground part (seedling) of the Zea mays L. plant in soil with divalent mercury; and D shows the dry weight and fresh weight of the underground part (root) of the Zea mays L. plant in soil with divalent mercury;

FIG. 6 shows the tolerance of strains to methylmercury and divalent mercury, where A shows the cultivation in a 1/2SDY liquid medium without methylmercury; B shows the cultivation in a 1/2SDY liquid medium with 0.1 mg/L; C shows the cultivation in a 1/2SDY liquid medium with 0.2 mg/L methylmercury; D shows the cultivation in a 1/2SDY liquid medium with 10 mg/L divalent mercury; E shows the cultivation in a 1/2SDY liquid medium with 15 mg/L divalent mercury; F shows the cultivation in a 1/2SDY liquid medium with 20 mg/L divalent mercury; and data from left to right in each group of the figure are for WT, ΔMmd, C-ΔMmd, ΔMir, C-ΔMir, and ΔMmd::ΔMir, respectively;

FIG. 7 shows the tolerance of mycelium to methylmercury, with a scale bar of 7 mm;

FIG. 8 shows the tolerance of mycelium to divalent mercury, with a scale bar of 7 mm;

FIG. 9 shows the Michaelis-Menten equation diagram of the MMD enzymatic reaction, and the Michaelis constant Km and the maximum reaction rate Vmax calculated by double reciprocal plot.

### DETAILED DESCRIPTION OF THE EMBODIMENTS

The present disclosure provides a fungus for treating mercury contamination, where the fungus expresses an MMD and an MIR; and the fungus includes a Metarhizium fungus and a non-Metarhizium fungus, and the non-Metarhizium fungus includes Fusarium oxysporum, Oidiodendron maius, Pyronema omphalodes, Amorphotheca resinae, Cadophora malorum, Hyaloscypha bicolor, Pseudogymnoascus sp, and Exophiala oligosperma.

The fungus of the present disclosure may include a Metarhizium fungus and a non-Metarhizium fungus, where the Metarhizium fungus expresses MMD and MIR; and the non-Metarhizium fungus includes a homologous protein of the MMD and a homologous protein of the MIR.

The Metarhizium fungus of the present disclosure may preferably include M. robertsii, M anisopliae, M. brunneum, M. guizhouense, M. majus, and M. acridum. The 6 Metarhizium fungi were deposited in the Agricultural Research Service Collection of Entomopathogenic Fungal Cultures (ARSEF), which belongs to the Agricultural Research Service Culture Collection (NRRL) of the United States (1815 N. University Street Peoria, IL 61604), with accession numbers of ARSEF2575, ARSEF549, ARSEF3297, ARSEF977, ARSEF297, and ARSEF324, respectively. The Metarhizium fungi can be inquired through the website: http://arsef.fpsnl.comell.edu/4DACTION/W_Search/Accessions.

The 8 non-Metarhizium fungi of the present disclosure may include Fusarium oxysporum, Cadophora malorum, Oidiodendron maius, Hyaloscypha bicolor, Pseudogymnoascus sp, Pyronema omphalodes, Exophiala oligosperma, and Amorphotheca resinae. The Fusarium oxysporum has an accession number of NRRL32931, which can be inquired through the website: https://nrrl.ncaur.usda.gov/cgi-bin/usda/fungi/results_public.ht?mv_action=back&mv_click=quer y_sort&sfd=substrate %2clocation_detail %2ccountry; the Cadophora malorum has an accession number of Bio-12245 (purchased from Beijing Baioubowei Biotechnology Co., Ltd., https://www.biobw.org/China-strain/bio-12245.html); and the Amorphotheca resinae has an accession number of Bio-104132 and an original number of CBS 186.54, which came from Netherlands and was purchased from Beijing Baioubowei Biotechnology Co., Ltd. (https://www.biobw.org/China-strain/bio-104132.html).

In the present disclosure, the above-mentioned 6 Metarhizium fungi and 8 non-Metarhizium fungi can remove methyl on methylmercury and can reduce divalent mercury.

In the present disclosure, a genome of the M. robertsii can express MMD and MIR, where a gene encoding the MMD may have a Genbank accession number of XP_007825874; and a gene encoding the MIR may have a Genbank accession number of XP_007824121. The MerB enzyme functions of some bacteria have been verified, such as Alphaproteobacteria Xanthobacter autotrophicus. However, the similarity between MMD and MerB of these bacteria with known functions is very low. For example, basic local alignment search tool (BLASTP) analysis of national center for biotechnology information (NCBI) shows that the highest similarity between MMD and the Alphaproteobacteria MerB gene (WP_159587663) is 33.85% (le⁻⁰⁹), and a homologous gene of a gene of the bacterial MerB most similar to MMD is a functionally unanalyzed gene from Actinobaceria bacterium (hypothetical protein, accession number: MB00836585), with a similarity of 41.99% (6e⁻⁶²). An MMD gene of M. robertsii has a similarity of 96.1% (e value: 0) to a homologous gene of M. brunneum (XP 014548844), a similarity of 96.1% (e value: 0) to a homologous gene of M. anisopiae (KFG84668), a similarity of 94.04% (e value: 0) to a homologous gene of M. majus (KIE02702), a similarity of 93.93% (e value: 0) to a homologous gene of M guizhouense (KID85335), and a similarity of 74.4% (e⁻³⁸) to a homologous gene of M. acridum (XP_007815236). The MMD gene has a similarity of 65.02% (9e¹³⁵) to a homologous gene of Fusarium oxysporum, a similarity of 51.96% (e⁻⁹⁷) to a homologous gene of Cadophora malorum, a similarity of 50.18% (2e⁻⁸⁹) to a homologous gene of Oidiodendron maius Zn, a similarity of 60% (4e⁻²⁹) to a homologous gene of Hyaloscypha bicolor E, a similarity of 27.98% (9e⁻¹⁰) to a homologous gene of Pyronema omphalodes, a similarity of 27.27% (2e⁻⁰⁷) to a homologous gene of Exophiala oligosperma, a similarity of 29.24% (3e⁻⁰⁶) to a homologous gene of Pseudogymnoascus destructans, and a similarity of 26.22% (2e⁻⁰⁵) to a homologous gene of Amorphotheca resinae.

In an embodiment of the present disclosure, an MMD coding gene Mmd-knockout mutant ΔMmd of M. robertsii and a complementary strain C-ΔMmd thereof are constructed. Compared with the wild-type strain, the ability of the mutant ΔMmd to eliminate methylmercury in the environment is significantly decreased, and more methylmercury is accumulated in the hyphae. The MMD protein is expressed and purified in Escherichia coli (E. coli). The MMD protein can remove methyl on methylmercury to produce divalent mercury. The MMD has become the first reported fungal MMD. In the present disclosure, similarity analysis is conducted for MMD, which includes the PFAM03243 domain in the bacterial alkylmercury lyase MerB. However, the MMD of M. robertsii has a very low similarity to the bacterial MerB.

In some bacteria, in addition to the MerB protein for methylmercury demethylation, there is divalent mercury reductase MerA, which constitute an operon. With the bacterial MerA as a query, a homologous gene (Genbank accession number: XP_007824121) is discovered in M. robertsii through the BLASTP and named MIR. Although no fungal divalent mercury reductase has been reported, homologous genes of MIR are widely present in fungi. A bacterial divalent mercury reductase with the highest similarity to M. robertsii MIR is encoded by a gene of Chloroflexi bacterium (Genbank accession number: MBN9390035), with a similarity of 55.49% (e value: 0). Similar to the Mmd gene, the knockout of the Mir gene can significantly reduce the divalent mercury reducing activity of M. robertsii, and the MIR has become the first reported fungal MIR. Biochemical analysis shows that the MIR protein expressed and purified in E. coli has the ability to reduce divalent mercury into nonvalent mercury.

The present disclosure also provides a microbial agent for methylmercury demethylation and divalent mercury reduction, including at least one of the fungi described above.

Any one or a combination of two or more of the Metarhizium fungus and 8 non-Metarhizium fungi in the present disclosure can be used to remove methyl in methylmercury and reduce divalent mercury. Therefore, the Metarhizium fungus and 8 non-Metarhizium fungi can be used to prepare a microbial agent.

The present disclosure has no specific limitations on a preparation method of the microbial agent, and a conventional fungal cultivation method in the art may be adopted. In the embodiments, Metarhizium is taken as an example for illustration, but it should not be regarded as the full protection scope of the present disclosure. The preparation method may preferably include: allowing spores of the Metarhizium fungus cultivated on potato dextrose agar (PDA) for 14 d to be uniformly suspended in a 0.01% (v/v) TritonX-100 aqueous solution to prepare a spore suspension with a concentration of 1×10⁸ spores/mL; and inoculating 1×10⁸ spores into an SDY medium (Sabroud dextrose broth plus 1% yeast extract) and cultivating for 36 h, and collecting hyphae through vacuum filtration in a sterile environment.

The present disclosure also provides use of any one or a combination of two or more of the Metarhizium fungus and 8 non-Metarhizium fungi, or the microbial agent in the removal of mercury contamination.

The present disclosure also provides a filter element for methylmercury demethylation and divalent mercury reduction, where the filter element uses hyphae of at least one of the fungi described above as a filler.

In the present disclosure, the above-mentioned hyphae can be used as a filler to prepare a filter element for removing methylmercury and divalent mercury in water. The present disclosure has no specific limitations on a preparation method and specifications of the filter element, and a conventional method in the art can be adopted.

The present disclosure also provides a filter device for removing methylmercury and divalent mercury in water, including the filter element.

In the filter device of the present disclosure, multiple filter elements can be arranged in series to ensure a filtering effect; or a single filter element can be arranged, such that a liquid reciprocally passes through the filter element. The present disclosure has no specific limitations on a specific shape and structure of the filter device.

The present disclosure also provides a method for removing methylmercury and divalent mercury in water, including the following steps: placing the microbial agent in the water and stirring for more than 48 h, or filtering the water through the filter element or the filter device.

In the present disclosure, the hyphae may preferably be placed in water to be treated and stirred at 26° C., and a rate for the stirring may preferably be 100 rpm. After the stirring is conducted for 48 h, a significant methylmercury and divalent mercury removal effect can be achieved. The water of the present disclosure may preferably include freshwater or seawater. In the present disclosure, a volume-to-mass ratio of the water to the microbial agent may preferably be 20 mL: 0.2 g (wet weight). At a methylmercury concentration of 1 mg/L, methylmercury in the water can be completely removed; and at a methylmercury concentration of 5 mg/L, the Metarhizium hypha treatment can still remove about 50% to 70% of methylmercury in the water. At a divalent mercury concentration of 10 mg/L, the Metarhizium hypha treatment can still remove about 56% of divalent mercury in the water. In an embodiment of the present disclosure, three of the 8 non-Metarhizium fungi are taken as examples for illustration, including Fusarium oxysporum, Cadophora malorum, and Amorphotheca resinae, where the former two include a homologous gene with the highest similarity to a coding gene of MMD of M. robertsii, and the Amorphotheca resinae includes a homologous gene with the lowest similarity to the coding gene of MMD. These three non-Metarhizium fungi can also remove methylmercury and divalent mercury in freshwater or seawater. In freshwater with a methylmercury concentration of 50 μg/L, Fusarium oxysporum, Cadophora malorum, and Amorphotheca resinae can remove 90%, 95%, and 97% of methylmercury in the freshwater, respectively; and in seawater with the same methylmercury concentration, Fusarium oxysporum, Cadophora malorum, and Amorphotheca resinae can remove 90%, 95%, and 94% of methylmercury in the seawater, respectively. That is, methylmercury in the freshwater or seawater of this methylmercury concentration can be basically removed, leaving only a trace amount methylmercury. In freshwater or seawater with 10 mg/L of divalent mercury, the three fungal strains can remove about 50% of divalent mercury in the freshwater; and the three fungal strains can remove 55% to 60% of divalent mercury in the seawater.

The present disclosure can also directly pass the water through the filter element or the filter device. In an embodiment, through simulation, the microbial agent is filled into a glass column with a diameter of 3 cm to construct a filter device with hyphae as a matrix. The device can be used to treat tap water with 100 μg/L methylmercury or 10 mg/L Hg²⁺. When a flow rate of the filter device is set to 0.1 mL/min, after 30 mL of tap water with 100 μg/L methylmercury is subjected to the first filtration, 80% of methylmercury remains in the tap water, and after the tap water is subjected to the second filtration, methylmercury in the tap water is basically completely removed. At the same flow rate, after 30 mL of tap water with 10 mg/L of Hg²⁺ is subjected to the first filtration, a divalent mercury content in the tape water is decreased by 60%, the divalent mercury content is decreased by 67% after the second filtration, and the divalent mercury content is decreased by 80% after the third filtration.

The present disclosure also provides a method for removing methylmercury and divalent mercury in soil, including the following steps: planting a plant that has a symbiotic relationship with the fungus in the soil, and inoculating the fungus. In an embodiment of the present disclosure, the inoculation of Metarhizium is taken as an example for illustration. After the Metarhizium is inoculated in the soil, the accumulation of methylmercury and divalent mercury in a plant is significantly reduced; and compared with a plant not inoculated with the Metarhizium, a methylmercury content in the plant inoculated with Metarhizium is decreased by 2.58 times, where a methylmercury content in the aboveground part is decreased by 2 times and a methylmercury content in the underground part is decreased by 2.52 times, and similarly, a divalent mercury content in the plant is decreased by 4.19 times, where a divalent mercury content in the aboveground part is decreased by 3 times and a divalent mercury content in the underground part is decreased by 6.2 times. After the Metarhizium is inoculated, a methylmercury content in the rhizosphere soil of the plant is decreased by 1.2 times, and a divalent mercury content in the rhizosphere soil is decreased by 1.1 times.

The plant of the present disclosure may preferably include a gramineous plant such as Zea mays L. and/or Pennisetum purpureum (P. purpureum) and a woody plant such as Morus alba L. and/or Acer spp. More specifically, the herbaceous plant may include P. purpureum and/or Zea mays L., and the woody plant may preferably include Morus alba L. and/or Acer spp. The present disclosure has no specific limitations on a method, a row spacing, and the like for the planting, and a conventional planting method in the art may be adopted.

The inoculation of the present disclosure may preferably include: irrigating roots once with a spore suspension of the Metarhizium fungus, where roots of each plant are irrigated with 10 mL of the spore suspension (1×10⁵ spores/mL).

The present disclosure also provides a method for identifying a fungus capable of methylmercury demethylation, including the following step: analyzing and comparing homologous proteins of MMD of the Metarhizium fungus by the BLASTP provided by NCBI to find other fungi with a homologous protein of MMD.

The fungus and the microbial agent for treating mercury contamination, the use thereof, the mercury removal method, and the method for identifying a fungus capable of treating mercury contamination provided by the present disclosure are described in detail below with reference to examples, but the examples cannot be understood as limiting the protection scope of the present disclosure.

### Example 1 Analysis of the Ability of 6 Metarhizium Fungi and 3 Non-Metarhizium Fungi to Remove Methyl in Methylmercury and Remove Divalent Mercury in a Medium

Cultivation and preparation of hyphae: Spores of Metarhizium (M. robertsii, M anisopliae, M. brunneum, M. guizhouense, M. majus, M. acridum) cultivated on PDA for 14 d were uniformly suspended in a 0.01% TritonX-100 aqueous solution to prepare a spore suspension with a concentration of 1×10⁸/mL; and 1×10⁸ spores were inoculated into an SDY medium (Sabouraud liquid medium, with 1% yeast extract) and cultivated for 36 h, and hyphae were collected through vacuum filtration in a sterile environment. From the above-mentioned 8 non-Metarhizium fungi, 3 were selected to analyze the ability to remove methyl in methylmercury and remove divalent mercury, where Fusarium oxysporum and Cadophora malorum included a homologous gene with the highest similarity to MMD of M. robertsii, and Amorphotheca resinae included a homologous gene with the lowest similarity to MMD. In addition, Beauveria bassiana (B. bassiana) and Saccharomyces cerevisiae (S. cerevisiae) without a homologous gene of MMD were used as negative controls. A preparation method of fungal hyphae was the same as the preparation method of the Metarhizium hyphae.

S. cerevisiae cultivation: An empty control strain BY4741 was inoculated on a plate with a yeast peptone mannitol (YPM) medium and cultivated at 30° C. for 3 d to 4 d, and then single colonies were picked and inoculated in a corresponding liquid medium, and cultivated at 30° C. and 220 rpm for 16 h to 24 h, at which point an OD₆₀₀ reached 1.0 to 1.5. A concentration of a bacterial solution was adjusted to OD₆₀₀ nm of 1, and 1 mL of the bacterial solution was taken and centrifuged (800 rpm, 10 min) to collect yeast; then the yeast were resuspended in the same volume of a liquid medium with methylmercury, treated at 220 rpm for 24 h, and centrifuged to obtain a supernatant and yeast; and the degradation of methylmercury was detected.

Treatment of methylmercury by hyphae: The hyphae prepared above (with a wet weight of 0.2 g) were transferred to 20 mL of an SDY liquid medium with 50 μg/L of methylmercury (in a 50 mL Erlenmeyer flask), and then cultivated for 48 h (26° C., 100 rpm) after the hyphae were uniformly dispersed, and a supernatant and hyphae were each collected by vacuum filtration.

Analysis of total mercury content in the hyphae and supernatant: The supernatant and hypha were each lyophilized, then 5 mL of concentrated nitric acid (6 M) was added to treat at 110° C. for 2 h, and ultrapure water (UPW) was added to a total volume of 50 mL; and then inductively coupled plasma mass spectrometry (ICP-MS) (PerkinElmer NexION 300X, Agilent Technologies 7800) was used to detect Hg ions in the sample to obtain the total mercury content.

ICP-MS conditions: radio frequency (RF) power: 1,550 w; sprayer (Perfluoroalkoxy) PFA: 100 L/min; atomizing chamber: quartz; Scott dual channel; sampling depth: 4.5 mm; carrier gas flow rate: 0.75 L/min; and makeup gas flow rate: 0.4 L/min.

High-performance liquid chromatography (HPLC)-ICP-MS [Agilent Infinity 1260 II (HPLC) and Agilent Technologies 7800 ICP-MS (ICP-MS)] was used to detect methylmercury and divalent mercury contents, where ICP-MS analysis conditions were as described above. HPLC conditions: mobile phase [a solution A (an aqueous solution of 10 mmol/L ammonium acetate and 0.12% L-cysteine, pH 7.5) and a solution B (methanol) were mixed in a ratio of 92:8]; chromatographic column: Zorbax Eclipse Plus C-18 150 mm×4.6 mm (with an inner diameter of 5 μm); and gradient elution at a flow rate of 1 mL/min. In order to detect methylmercury and divalent mercury in the supernatant, the supernatant was first diluted 10 times with the mobile phase and filtered through a 0.22 μm filter membrane, and then analyzed by HPLC-ICP-MS.

In order to detect methylmercury and divalent mercury in the mycelium, the mycelium was treated overnight in 5 mL of concentrated hydrochloric acid (6 M) and then treated for 60 min in an ultrasonic bath at room temperature, then UPW was added to a final volume of 50 mL, and a resulting mixture was thoroughly mixed and then analyzed by HPLC-ICP-MS.

Results were shown in Table 1. After the SDY medium with methylmercury (50 μg/L) was treated for 48 h by hyphae of the 6 Metarhizium fungi, no methylmercury was detected in culture supernatants of 3 Metarhizium fungi (M. robertsii, M. guizhouense, and M. brunneum), and only a trace amount of methylmercury was detected in culture supernatants of M. acridum, M. anisopliae, and M. majus. No methylmercury was detected in culture mycelia of M guizhouense and M. brunneum, and a trace amount of methylmercury was detected in culture mycelia of 4 Metarhizium fungi (M. robertsii, M. anisopliae, M. majus, and M. acridum) (Table 1). A trace amount of divalent mercury was detected in a culture supernatant of M. robertsii, and the total mercury contents of the 6 fungi were similar to that in the negative control without fungi (Table 1). A specified amount of divalent mercury could be detected in the culture supernatants and mycelia of the 6 Metarhizium fungi, where M. robertsii had the highest divalent mercury content and M. anisopliae had the lowest divalent mercury content (Table 1).

After the SDY medium with methylmercury (50 μg/L) was treated for 48 h with non-Metarhizium fungi, only a trace amount of methylmercury was detected in culture supernatants of Cadophora malorum and Amorphotheca resinae, and the production of divalent mercury was detected. 17.5% of methylmercury still remained in the culture supernatants of Fusarium oxysporum NRRL32931, but both of the fungi had the ability to degrade methylmercury (Table 1). However, B. bassiana and S. cerevisiae had no ability to degrade methylmercury (Table 1).

TABLE 1
Analysis of the ability of 6 Metarhizium fungi and 3 non-Metarhizium fungi, and B. bassiana
and S. cerevisiae without an MMD homologous gene to remove methyl in methylmercury

| 50 μg/L of methylmercury | | | | | | | Total |
|---|---|---|---|---|---|---|---|
| is added to 20 mL of SDY | Methylmercury content (ng) | | | Divalent mercury content (ng) | | | mercury |
| (Total: 1,000 ng) | Supernatant | Mycelium | Sum | Supernatant | Mycelium | Sum | (ng) |
| Metarhizium robertsii | ND | 5 ± 1.5^{e} | 5 ± 1.5^{e} | 108.4 ± 14.5^{b} | 7.5 ± 1.3^{e} | 115.9 ± 15.7^{c} | 795 ± 30^{a} |
| Metarhizium guizhouense | ND | ND | ND | 40.4 ± 4.1^{c} | 7.5 ± 2.6^{e} | 47.9 ± 2.3^{e} | 741 ± 44^{a} |
| Metarhizium brunneum | ND | ND | ND | 29.1 ± 1.2^{d} | 15.7 ± 2.9^{d} | 44.8 ± 3.4^{e} | 674 ± 30^{a} |
| Metarhizium anisopliae | 7.7 ± 0.7^{g} | 5.0 ± 0.6^{e} | 12.7 ± 1.64^{d} | 19.6 ± 1.1^{e} | 3.5 ± 0.3^{f} | 23.1 ± 1.2^{f} | 759 ± 6^{a} |
| Metarhizium acridum | 12.8 ± 1.2^{f} | 12.9 ± 0.9^{d} | 25.8 ± 0.9^{c} | 35.4 ± 1.5^{cd} | 32.7 ± 5.6^{c} | 68.1 ± 7.1^{d} | 767 ± 19^{a} |
| Metarhizium majus | 6.0 ± 1.1^{g} | 3.7 ± 0.42^{e} | 9.7 ± 1.5^{de} | 31.3 ± 2.0^{cd} | 11.3 ± 0.9^{e} | 42.6 ± 2.8^{e} | 753 ± 8^{a} |
| Amorphotheca resinae | 40.3 ± 14^{e} | 145.5 ± 10.5^{c} | 185.8 ± 24.5^{b} | 166 ± 6.7^{a} | 127 ± 6.0^{b} | 293 ± 2.3^{b} | 790.3 ± 17.7^{a} |
| Cadophora malorum | 92.4 ± 7.0^{d} | 128.5 ± 13.5^{c} | 220.9 ± 20.5^{b} | 154 ± 14.2^{a} | 149 ± 9.0^{a} | 303 ± 21.5^{ab} | 791 ± 12.3^{a} |
| Fusarium oxysporum | 115 ± 10^{d} | 112.5 ± 14.5^{c} | 228 ± 5^{b} | 195.7 ± 5.2^{a} | 165 ± 7.5^{a} | 360 ± 10.9^{a} | 810.7 ± 17.9^{a} |
| Beauveria bassiana | 330.1 ± 21^{c} | 390.5 ± 11^{a} | 721.5 ± 31.1^{a} | ND | ND | ND | 750 ± 10.5^{a} |
| Saccharomyces cerevisiae | 528 ± 57.5^{b} | 283 ± 68^{b} | 811 ± 68^{a} | ND | ND | ND | 793 ± 20.5^{a} |
| Untreated with a fungus | 813.5 ± 34^{a} | — | 813.5 ± 34.5^{a} | ND | ND | ND | 814 ± 14.6^{a} |

ND indicates that it is not detected.
“—” (The horizontal line) indicates that the assay was not conducted. Within each column, values with different letters are significantly different (P < 0.05, Tukey's test in one-way ANOVA). The table legend is the same in other tables.

Treatment of divalent mercury by hyphae: The hyphae prepared above (with a wet weight of 0.2 g) were transferred to 20 mL of an SDY liquid medium with 10 mg/L of divalent mercury (in a 50 mL Erlenmeyer flask), and then cultivated for 48 h (26° C., 100 rpm) after the hyphae were uniformly dispersed, and a supernatant and hyphae were each collected by vacuum filtration. The subsequent treatment and detection steps were the same as that of the treatment of methylmercury by hyphae.

Results were shown in Table 2. After the SDY medium with divalent mercury (10 mg/L) was treated for 48 h with the hyphae of the 6 Metarhizium fungi, divalent mercury contents in culture supernatants of the 6 Metarhizium fungi were all decreased by nearly 60% relative to uninoculated control, and there was no significant difference in the ability to remove divalent mercury among the 6 Metarhizium fungi. Divalent mercury contents detected in culture hyphae of M. brunneum and M. anisopliae were significantly lower than that of the other 4 Metarhizium fungi, and the total mercury contents of the 6 fungi were similar to that in the negative control without fungi (Table 2).

After the SDY medium with divalent mercury (10 mg/L) was treated for 48 h with non-Metarhizium fungi, divalent mercury contents in culture supernatants of 3 non-Metarhizium fungi were decreased to varying degrees relative to that of the uninoculated control, and there were significant differences in the ability to remove divalent mercury among different species of fungi, where Amorphotheca resinae removed 60% of divalent mercury in water, and Fusarium oxysporum and Cadophora malorum removed about 50% of divalent mercury in water. There was no significant difference in the total mercury content among the 3 fungi (Table 2).

TABLE 2
Analysis of the ability of the 6 Metarhizium fungi and
3 non-Metarhizium fungi to remove divalent mercury

| 10 mg/L of divalent | | | | |
|---|---|---|---|---|
| mercury is added to 20 | Divalent mercury content (μg) | | | Total mercury |
| mL of SDY (Total: 200 μg) | Supernatant | Mycelium | Sum | (μg) |
| Metarhizium robertsii | 73.3 ± 1.5^{c} | 42.9 ± 1.5^{cd} | 116.2 ± 1.8^{c} | 172.3 ± 6.1^{a} |
| Metarhizium | 77.5 ± 0.6^{c} | 38.1 ± 1.4^{de} | 115.6 ± 1.9^{c} | 161.7 ± 1.2^{a} |
| guizhouense | | | | |
| Metarhizium brunneum | 67.1 ± 0.7^{c} | 34.5 ± 0.7^{e} | 101.6 ± 0.1^{d} | 171.1 ± 1.2^{a} |
| Metarhizium anisopliae | 71.3 ± 1.3^{c} | 26.9 ± 0.4^{g} | 98.3 ± 1.3^{d} | 172.6 ± 4.9^{a} |
| Metarhizium acridum | 71.6 ± 2.2^{c} | 44.0 ± 1.2^{f} | 115.6 ± 3.4^{c} | 162.9 ± 1.2^{a} |
| Metarhizium majus | 71.2 ± 3.4^{c} | 44.9 ± 0.9^{c} | 116.1 ± 2.6^{c} | 161.2 ± 2.7^{a} |
| Amorphotheca resinae | 82.6 ± 5.3^{b} | 36.8 ± 2.4^{de} | 126.3 ± 5.4^{c} | 178.3 ± 6.1^{a} |
| Cadophora malorum | 71.6 ± 4.0^{c} | 54.8 ± 2.2^{b} | 153.5 ± 2.6^{b} | 174.7 ± 2.2^{a} |
| Fusarium oxysporum | 84.1 ± 2.0^{b} | 69.3 ± 0.6^{a} | 153.9 ± 3.3^{b} | 181.1 ± 6.4^{a} |
| Untreated with a fungus | 175.3 ± 15.6^{a} | — | 175.3 ± 15.6^{a} | 182.6 ± 5.4^{a} |

### Example 2 Removal of Methylmercury and Divalent Mercury Contamination in Freshwater and Seawater by Mycelia of the Metarhizium Fungus and 3 Non-Metarhizium Fungi

Experimental procedure: M. robertsii was taken as a representative of Metarhizium. From the 8 non-Metarhizium fungi, Fusarium oxysporum, Cadophora malorum, and Amorphotheca resinae were selected as representatives, where the former two included a homologous gene with the highest similarity to a coding gene of MMD of M. robertsii, and the Amorphotheca resinae included a homologous gene with the lowest similarity to the coding gene of MMD. The above fungi were each cultivated in an SDY medium to obtain hyphae, and the hyphae were used to treat methylmercury in nutrient-free freshwater (tap water) and seawater (tap water with 2.24% sea salt (Red sea Fish Pharm Ltd.)).

The hyphae prepared above (with a wet weight of 0.2 g) were transferred to 20 mL of freshwater or seawater with 0.05 mg/L, 0.5 mg/L, 1 mg/L, 2 mg/L, and 5 mg/L of methylmercury to treat for 48 h at 26° C. under slight shaking (100 rpm), and then a methylmercury content in the water was analyzed according to the above method.

Results were shown in Table 3. After the 48 h of treatment, methylmercury in tap water with methylmercury concentrations of 0.05 mg/L, 0.5 mg/L, and 1 mg/L was basically completely removed; 12% and 50% of methylmercury still remained in tap water with methylmercury concentrations of 2 mg/L and 5 mg/L, respectively (Table 3); and the total mercury content was not significantly different from that of the uninoculated control. Similarly, after 48 h of treatment, methylmercury in seawater with a methylmercury concentration of 0.05 mg/L was completely completed; methylmercury in seawater with methylmercury concentrations of 0.5 mg/L and 1 mg/L was basically completely removed, and only a trace amount of methylmercury was detected; and after 48 h of the treatment, 64% and 43% of methylmercury still remained in seawater with methylmercury concentrations of 2 mg/L and 5 mg/L, respectively (Table 3).

The hyphae prepared above (with a wet weight of 0.2 g) were transferred to 20 mL of tap water or seawater with 0.5 mg/L, 1 mg/L, 2 mg/L, 5 mg/L or 10 mg/L of divalent mercury to treat for 48 h at 26° C. under slight shaking (100 rpm), and then a divalent mercury content in the water was analyzed according to the above method.

Results were shown in Table 4. After 48 h of the treatment, divalent mercury in tap water or seawater with a divalent mercury concentration of 0.5 mg/L was basically completely removed; more than 70% of divalent mercury in tap water or seawater with a divalent mercury concentration of 1 mg/L was removed; and 50% of divalent mercury in tap water or seawater with divalent mercury concentrations of 5 mg/L and 10 mg/L was removed.

Results were shown in Table 5. The 3 non-Metarhizium fungi all have the ability to remove methylmercury and divalent mercury in freshwater or seawater.

TABLE 3
Analysis of the ability of M. robertsii to
remove methylmercury in freshwater or seawater

| Methylmercury is added | | | | |
|---|---|---|---|---|
| to 20 mL of water at | Methylmercury content in | | Methylmercury content in | |
| different concentrations | freshwater supernatant (μg) | | seawater supernatant (μg) | |
| Methylmercury | Treated with | Untreated | Treated with | Untreated |
| concentration (mg/L) | mycelium | control | mycelium | control |
| 0.05 (total: 1 μg) | ND | 0.832 ± 0.05 | ND | 0.8 ± 0.01 |
| 0.5 (total: 10 μg) | ND | 8.0 ± 2.3 | ND | 8.3 ± 1.5 |
| 1 (total: 20 μg) | ND | 18.3 ± 2.2 | 0.8 ± 0.1^{b} | 18.6 ± 2.3^{a} |
| 2 (total: 40 μg) | 19.6 ± 1.6^{b} | 35.7 ± 5.2^{a} | 20.7 ± 3.4^{b} | 32.5 ± 4.7^{a} |
| 5 (total: 100 μg) | 40.5 ± 1.4^{b} | 82.3 ± 2.40^{a} | 37.0 ± 1.0^{b} | 85.3 ± 0.9^{a} |

TABLE 4
Analysis of the ability of M. robertsii to remove
divalent mercury in freshwater or seawater

| Divalent mercury is added | | | | |
|---|---|---|---|---|
| to 20 mL of water at | Divalent mercury content in | | Divalent mercury content in | |
| different concentrations | freshwater supernatant (μg) | | seawater supernatant (μg) | |
| Divalent mercury | Treated with | Untreated | Treated with | Untreated |
| concentration (mg/L) | mycelium | control | mycelium | control |
| 0.5 (total: 10 μg) | ND | 8.0 ± 2.3 | ND | 9.1 ± 0.7 |
| 1 (total: 20 μg) | 4.3 ± 0.3^{b} | 18.3 ± 2.2^{a} | 5.2 ± 1.3^{b} | 19.3 ± 1.1^{a} |
| 2 (total: 40 μg) | 19.6 ± 1.6^{b} | 37.8 ± 3.4^{a} | 20.7 ± 3.4^{b} | 36.5 ± 4.5^{a} |
| 5 (total: 100 μg) | 41.34 ± 3.3^{b} | 89.4 ± 4.6^{a} | 42.6 ± 3.4^{b} | 91.6 ± 5.7^{a} |
| 10 (total: 200 μg) | 83.5 ± 1.6^{b} | 166.8 ± 3.3^{a} | 81.5 ± 3.4^{b} | 167.7 ± 1.9^{a} |

TABLE 5
Ability of non-Metarhizium fungi to remove methylmercury in freshwater or seawater

| 50 μg/L of | | | | | | | |
|---|---|---|---|---|---|---|---|
| methylmercury | | | | | | | |
| is added to 20 | | | | | | | Total |
| mL of water | Methylmercury content in freshwater(ng) | | | Methylmercury content in seawater (ng) | | | mercury |
| (Total: 1000 ng) | Supernatant | Mycelium | Sum | Supernatant | Mycelium | Sum | (ng) |
| A. resinae | 45.5 ± 2.6^{c} | 124.5 ± 6.2^{a} | 170.6 ± 5.6^{c} | 53.5 ± 1.6^{c} | 110 ± 3.5^{b} | 163.5 ± 2.2^{c} | 790.3 ± 17.7^{a} |
| C. malorum | 27.5 ± 2.7^{d} | 98.3 ± 5.9^{b} | 125.8 ± 7.7^{d} | 36.9 ± 1.8^{d} | 104.8 ± 3.6^{b} | 141.7 ± 5.2^{d} | 791 ± 12.3^{a} |
| F. oxysporum | 89.4 ± 4.2^{b} | 134.9 ± 11.0^{a} | 224.2 ± 8.7^{b} | 94.4 ± 2.3^{b} | 136.9 ± 4.7^{a} | 231.3 ± 3.6^{b} | 810.7 ± 17.9^{a} |
| Untreated with | 827.2 ± 26^{a} | — | 827.2 ± 26^{a} | 846.5 ± 25^{a} | — | 846.5 ± 25^{a} | 814 ± 14.6^{a} |
| a fungus | | | | | | | |

### Example 3 Removal of Methylmercury in Tap Water and Seawater by Mycelium-Filled Column Treatment

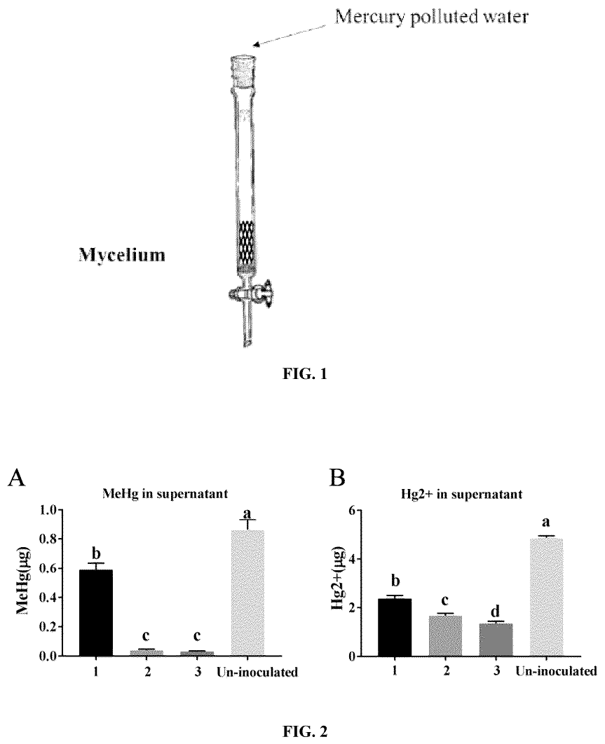

The hyphae obtained by cultivation in the SDY medium were filled into a glass column with a diameter of 3 cm (FIG. 1) to construct a filter with hyphae as a matrix. Tap water with 100 μg/L methylmercury or 10 g/L Hg²⁺ was allowed to pass through the hypha matrix at a flow rate of 0.1 mL/mm.

Results were shown in FIG. 2. In fresh water, After the first filtration, a methylmercury content in the tap water was decreased by 20%; after the second filtration, the methylmercury content was decreased by 20 times, that is, methylmercury was basically completely removed; and the third filtration did not further reduce the methylmercury content. The first filtration reduced a divalent mercury content in the tap water by 60%; the second filtration further reduced the divalent mercury content by 67%; and the third filtration reduced the divalent mercury content by 80%. In sea water, After the first filtration, a methylmercury content in the tap water was decreased by 20%; after the second filtration, the methylmercury content was decreased by 70%, and after the third filtration methylmercury was basically completely removed; The first filtration reduced a divalent mercury content in the tap water by 30%; the second filtration further reduced the divalent mercury content by 53%; and the third filtration reduced the divalent mercury content by 75%

### Example 4 Removal of Methylmercury and Divalent Mercury in Soil by the Planting of Plants and the Release of M. robertsii Spores

Zea mays L. plants were planted in soil with methylmercury, a spore suspension of Metarhizium was added to roots of the Zea mays L. plants, and after the Zea mays L. plants were cultivated for 14d, samples were collected to test a mercury form and a total mercury content in rhizosphere soil and plants.

1) Analysis of the Tolerance of Zea mays L. To Methylmercury and Divalent Mercury

Methylmercury was added to the soil, where five concentrations of 0 μg/kg, 2.5 μg/kg, 5 μg/kg, 7.5 μg/kg, and 10 μg/kg were set. Divalent mercury was added to the soil, where five concentrations of 0 mg/kg, 20 mg/kg, 30 mg/kg, 40 mg/kg, and 50 mg/kg were set. The soil was placed in a cultivation vessel (with a height of 14 cm and a diameter of 7 cm).

Seed disinfection and cultivation: Zea mays L. seeds were disinfected for 5 min in 1% sodium hypochlorite and then washed three times with sterile water (1 min each time), disinfected for 10 min with 15% H₂O₂ and then washed three times with sterile water (1 min each time), and then placed in a 2% water agar medium and vernalized overnight at 4° C. Then, the pretreated seeds were sown into the soil, and 10 P. purpureum seeds or 5 Zea mays L. seeds were sown in each vessel.

In the soil with 2.5 μg/kg methylmercury, a germination rate of the Zea mays L. seeds was still 100%, and in the soil with 10 μg/kg methylmercury, the germination rate was 80%. When Zea mays L. and Metarhizium were used to treat methylmercury, a methylmercury concentration was set to 10 μg/kg.

In the soil with 20 mg/kg divalent mercury, a germination rate of the Zea mays L. seeds was 100%; in the soil with 30 mg/kg divalent mercury, the germination rate was 80%; and in the soil with 40 mg/kg divalent mercury, the germination rate was only 60%. In the next experiment, when Zea mays L. and Metarhizium were used to treat divalent mercury, a divalent mercury concentration was set to 20 mg/kg.

Treatment of Methylmercury and Divalent Mercury in Soil by Zea mays L. And Metarhizium

10 μg/kg of methylmercury and 20 mg/kg of divalent mercury were each added to the soil, and Zea mays L. seeds that had been disinfected one day in advance were sown into the soil with methylmercury or divalent mercury. The seeds were cultivated at 25° C. under 16 h light and 8 h dark cycles. After the seeds were cultivated for 4 d, 10 mL of a Metarhizium spore suspension with a concentration of 1×10⁵/mL (total number of spores: 1×10⁶) was added. After 10 d of co-cultivation, plant samples and soil samples were collected separately. The soil samples included rhizosphere soil and non-rhizosphere soil around plant roots, and the plant samples included aboveground part (seedling) and underground part (root). The soil samples and plant samples were each lyophilized, then 5 mL of hydrochloric acid (6 M) was added to digest overnight, and then ultrasonic extraction was conducted at room temperature. Water was added to an extracted sample to 50 mL, and the sample was filtered through a 0.22 μm filter membrane and then tested by HPLC-ICP-MS. A test method of total mercury was as follows: 5 mL of nitric acid (6 M) was added to digest the sample at 110° C. for 1 h, then water was added to 50 mL, and a resulting mixture was filtered through a 0.22 μm filter membrane and then tested by ICP-MS.

Results were shown in Table 5 and Table 6. Metarhizium promoted the resistance of plants to methylmercury and divalent mercury, reduced the accumulation of methylmercury and divalent mercury in plants, and effectively removed methylmercury and divalent mercury in the soil. Metarhizium reduced the methylmercury and divalent mercury in the soil by 30% and 25% respectively, and reduced the methylmercury and divalent mercury in plants by 61% and 77% respectively.

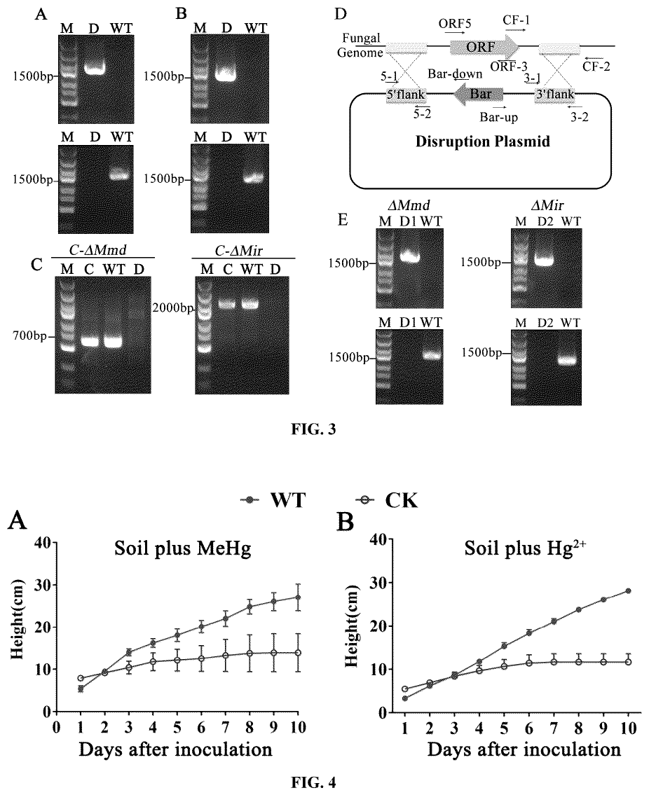

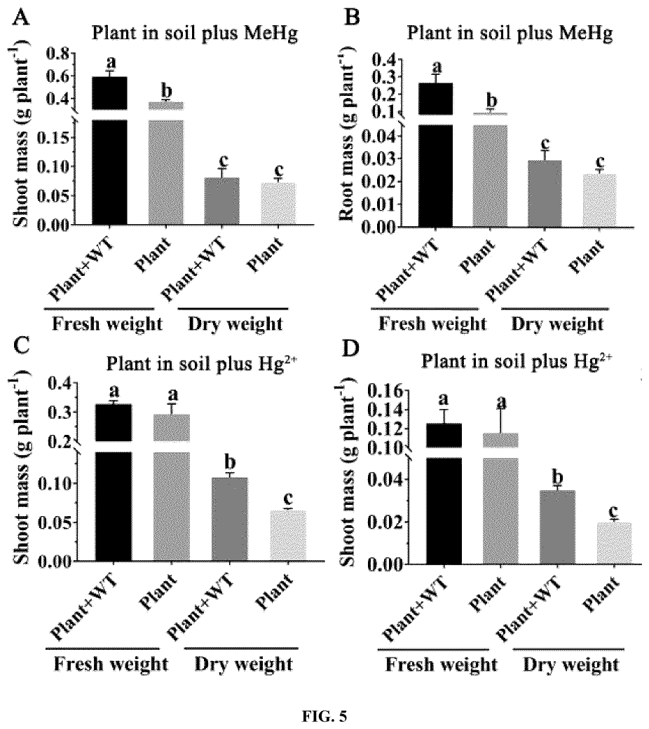

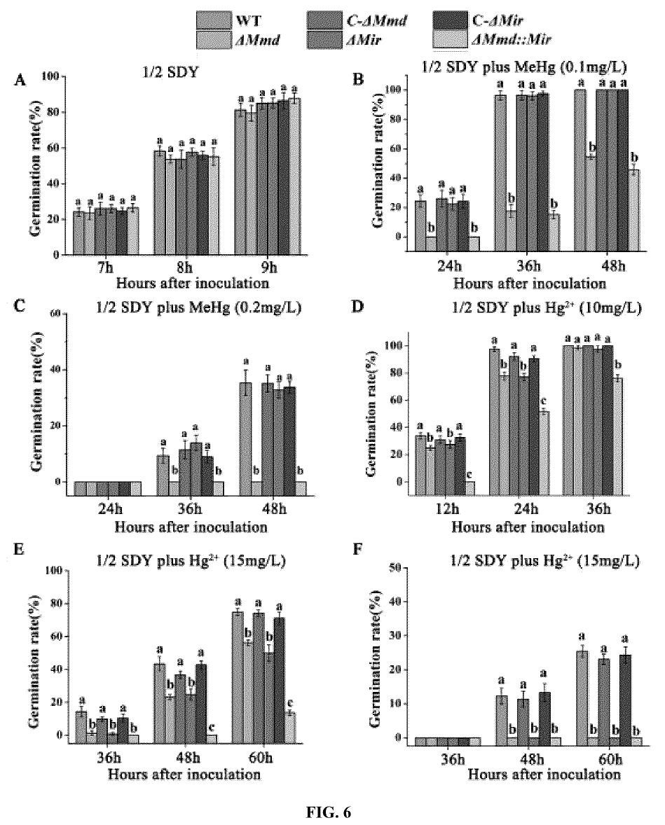

Physiological indexes of the plants, wet weights and dry weights of the aboveground parts (seedlings) and roots, and the daily growth rates of the plants inoculated with the spore suspension were determined. Results showed that a growth rate of the plant inoculated with the Metarhizium spores was significantly higher than that of the uninoculated plant (FIG. 4). In the soil with methylmercury, fresh weights of aboveground parts (seedlings) and roots of the WT spore-inoculated plants were significantly higher than that of the uninoculated plants; and in the soil with divalent mercury, dry weights of aboveground parts (seedlings) and roots of the WT spore-inoculated plants were significantly higher than that of the uninoculated plants (FIG. 5).

TABLE 5
Methylmercury and total mercury contents in soil and maize (Zea mays L.) plants

| 10 μg/kg of | Methylmercury content per g of dry weight (ng) | | | | | Total mercury per g of | |
|---|---|---|---|---|---|---|---|
| methylmercury | Rhizosphere | Non-rhizosphere | Aboveground | | Sum in | dry weight (ng) | |
| is added to soil | soil | soil | parts | Roots | plant | Plant | Soil |
| Plants treated | 6.6 ± 0.3^{b} | 9.3 ± 0.1^{a} | 247.5 ± 36.9^{b} | 803.0 ± 48.3^{b} | 503.4 ± 9.8^{b} | 105.4 ± 0.9^{a} | 8.7 ± 0.3^{a} |
| with WT spores | | | | | | | |
| Plants untreated | 8.1 ± 0.2^{a} | 9.4 ± 0.1^{a} | 494.9 ± 18.6^{a} | 2029.5 ± 76.1^{a} | 1298.5 ± 54.1^{a} | 113.1 ± 7.5^{a} | 8.1 ± 0.4^{a} |
| with the fungus | | | | | | | |
| Soil only | — | 9.6 ± 0.12^{a} | — | — | — | — | 9.2 ± 0.3^{a} |

TABLE 6
Divalent mercury and total mercury contents in soil and maize (Zea mays L.) plants

20 mg/kg of

| divalent | Divalent mercury content per g of dry weight (μg) | | | | | Total mercury per g | |
|---|---|---|---|---|---|---|---|
| mercury is | Rhizosphere | Non-rhizosphere | Aboveground | | Sum in | of dry weight (μg) | |
| added to soil | soil | soil | parts | Roots | plant | Plant | Soil |
| Plants treated | 14.7 ± 0.2^{b} | 19.2 ± 0.2^{a} | 13.1 ± 0.2^{b} | 7.5 ± 0.35^{b} | 10.8 ± 0.3^{b} | 19.5 ± 0.5^{a} | 19.7 ± 0.5^{a} |
| with WT spores | | | | | | | |
| Plants untreated | 17.2 ± 0.6^{a} | 19.2 ± 0.5^{a} | 40.4 ± 2.0^{a} | 46.8 ± 0.8^{a} | 42.9 ± 0.9^{a} | 20.3 ± 0.3^{a} | 17.8 ± 0.9^{a} |
| the fungus | | | | | | | |
| Soil only | — | 20.4 ± 0.4^{a} | — | — | — | — | 19.4 ± 0.3^{a} |

### Example 5 Function Study of MMD and MIR

### 1) Construction of Mutant Strains.

In order to study the functions of MMD and MIR, the present disclosure constructed MMD coding gene-knockout and MIR coding gene-knockout mutants ΔMmd and ΔMir and a double gene-knockout mutant ΔMmd::ΔMir based on homologous recombination and enzymatic digestion and ligation. Complementary strains C-ΔMmd and C-ΔMir of the mutants ΔMmd and ΔMir were also constructed. Primers used to construct the plasmids for the gene knockout were shown in Table 7.

The vectors used to construct Mmd single gene-knockout and Mir single gene-knockout mutants were pPk2-Bar-GFP-Mmd and pPk2-Bar-GFP-Mir, respectively, and the herbicide resistance gene Bar was adopted as a resistance gene. The Mmd single gene knockout vector was constructed by homologous recombination, and a construction method can be seen in the reference (Xu C, Zhang X, Qian Y, et al. A high-throughput gene disruption methodology for the entomopathogenic fungus M. robertsii. PLoS One. 2014; 9 (9): e107657. Published 2014 Sep. 15. doi: 10.1371/journal.pone.0107657). The Mir single gene knockout vector was constructed by enzymatic digestion and ligation, where a vector and a 5′ homology arm fragment were each digested with XbaI and ECORI and then ligated, and then the vector and a 3′ homology arm were each digested with DraI and then ligated.

A construction method of the Mmd and Mir double-knockout mutant (ΔMmd::ΔMir) was as follows: the Mir gene was further knocked out from the Mmd gene single-knockout mutant ΔMmd. Thus, a Mir gene knockout vector pPk2-NTC-GFP-Mir for the resistance gene NTC was constructed, and nourseothricin was adopted as a screening agent for all transformants (Zhang Q, Chen X, Xu C, et al. Horizontal gene transfer allowed the emergence of broad host range entomopathogens. Proc Natl Acad Sci USA. 2019; 116 (16): 7982-7989. doi: 10.1073/pnas.1816430116). A vector construction method was the same as that of the Mir single gene knockout vector.

Plasmids used in the construction of the complementary strains C-ΔMmd and C-ΔMir were pFBENGFP-gMmd and pFBENGFP-gMir, respectively, and the benomyl resistance gene was adopted as a resistance gene (Fang W, Pei Y, Bidochka M J. Transformation of M. anisopliae mediated by Agrobacterium tumefaciens. Can J Microbiol. 2006; 52 (7): 623-626. doi:10. 1139/w06-014). Fungal genetic transformation mediated by Agrobacterium tumefaciens (A. tumefaciens) was conducted according to the reference (Xu C, Zhang X, Qian Y, et al. A high-throughput gene disruption methodology for the entomopathogenic fungus M. robertsii. PLoS One. 2014; 9 (9): e107657. Published 2014 Sep. 15. doi: 10.1371/journal.pone.0107657). Verification results of each mutant and complementary strain were shown in FIG. 3.

TABLE 7
Primers for gene knockout, complementation, and verification

| | | SEQ | |
|---|---|---|---|
| | | ID | |
| Primer | Sequence | NO. | Use |
| DMmd-5-1 | GGGGACAGCTTTCTTGTACAAAGTGGATTTAGCAAACGACCA | 1 | Mmd gene knockout |
| DMmd-5-2 | GGGGACTGCTTTTTTGTACAAACTTGTCAGACTCAACAGCCA | 2 | |
| DMmd-3-1 | GGGGACAACTTTGTATAGAAAAGTTGTTTTGGTATGACCTGC | 3 | |
| DMmd-3-2 | GGGGACAACTTTGTATAATAAAGTTGTGAGACTTGGAATCGT | 4 | Verification of Mmd |
| DMmd-CF1 | AGGACTGGTGTTGGAGAG | 5 | knockout |
| DMmd-CF2 | CAGGTCATCGGATGAAG | 6 | |
| gMmd-5 | GGGCTAGCCTCCAAGACAGTACGTG | 7 | Complementation of |
| gMmd-3 | GGGATATCGAGCAACTGTGTACTTG | 8 | ΔMmd |
| cc-Mmd-5 | AAGATTCAACTGCTGACC | 9 | Verification of ΔMmd |
| cc-Mmd-3 | ACAAAGTTGACGACCAAG | 10 | complementation |
| DMir-5-1 | GGTCTAGACTGTTTGGCAGCATCAT | 11 | Mir knockout |
| DMir-5-2 | GGGAATTCTTGACATGGCCTATCAC | 12 | |
| DMir-3-1 | GGTTTAAATGGCTGAATGTAGCCGT | 13 | |
| DMir-3-2 | GGTTTAAACCTTCGAAGCTGGCCAT | 14 | |
| DMir-CF1 | GACAATATGGCTGAATG | 15 | Verification of Mir |
| DMir-CF2 | CGATCTCCGTTATGGTC | 16 | knockout |
| gMir-5 | GGACTAGTGCATAACTAAGGAAGTT | 17 | Complementation of |
| gMir-3 | GGGATATCCTATTTCAACGAGCCCC | 18 | ΔMir |
| cc-Mir-5 | TTATCACCGATAAAGGA | 19 | Verification of ΔMir |
| cc-Mir-3 | ATCCCAGAATCTTCTGC | 20 | complementation |
| E-Mmd-5 | GGGGATCCATGAGTCAACAGAGCCC | 21 | Prokaryotic expression |
| E-Mmd-3 | GGGAATTCTCAAGACAACCCGTACC | 22 | of Mmd |
| Bar-up | CGCCTGGACGACTAAACC | 23 | Screening markers for |
| Bar-down | TCAGCCTGCCGGTACCGC | 24 | verification of knockout |
| Sur-up | ATCGTGGAGTCATGTTTG | 25 | Screening markers for |
| Sur-down | CCAGTAAGTAATATATCC | 26 | verification of |
| | | | overexpression |
| NTC-up | CATCCACTGCACCTCAGAG | 27 | Screening markers for |
| NTC-down | GTACCGGCGGATGGGGTTC | 28 | verification of knockout |

### 2) Analysis of the Tolerance of Strains to Methylmercury and Divalent Mercury

In a 1/2SDY liquid medium, there was no difference in the spore germination rate among the mutant strains ΔMmd, ΔMir, and ΔMmd::ΔMir, the complementary strains C-ΔMmd and C-ΔMir, and the wild-type strain WT (FIG. 6A).

In a 1/2SDY liquid medium with 0.1 mg/L methylmercury, after cultivation for 12 h, spores of ΔMmd and ΔMmd::ΔMir did not germinate, while the strains WT, ΔMir, C-ΔMmd, and C-ΔMir had a spore germination rate of about 20%; and after cultivation for 36 h, almost all spores of the strains WT, ΔMir, C-ΔMmd, and C-ΔMir germinated, while only about 20% of spores of ΔMmd and ΔMmd::ΔMir germinated (B in FIG. 6).

In a 1/2SDY liquid medium with 0.2 mg/L methylmercury, spores of ΔMmd and ΔMmd::ΔMir failed to germinate, but after cultivation for 48 h, the strains WT, ΔMir, C-ΔMmd, and C-ΔMir had a spore germination rate of about 40% (C in FIG. 6).

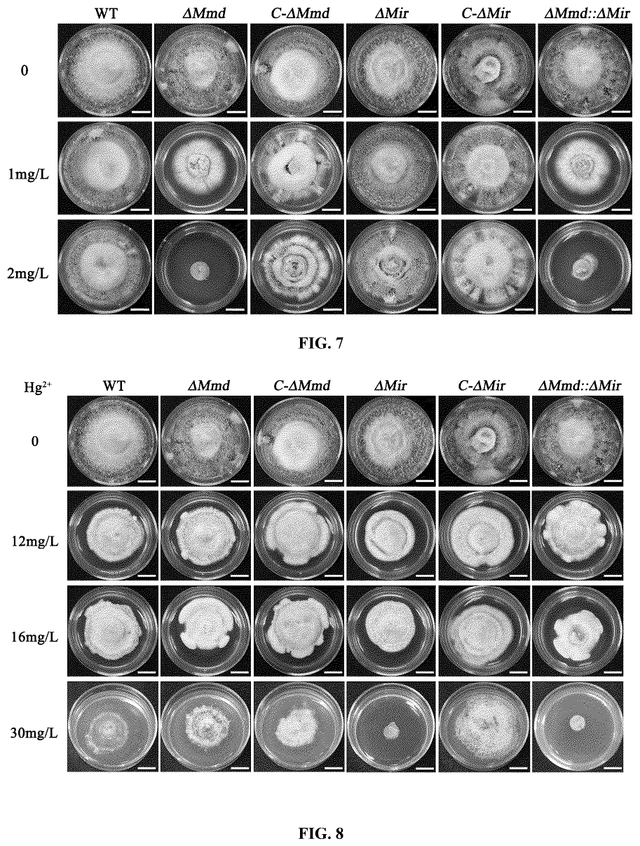

In a 1/2SDY liquid medium with 15 mg/L divalent mercury, after cultivation for 12 h, spores of ΔMmd, ΔMir, and ΔMmd::ΔMir basically did not germinate, and a germination rate of WT (15%) was not significantly different from that of C-ΔMmd and C-ΔMir; after cultivation for 24 h, spores of ΔMmd::ΔMir still did not germinate, and germination rates of ΔMmd and ΔMir were about 25%, which were significantly lower than that of WT, C-ΔMmd and C-ΔMir strains (40%); and after cultivation for 48 h, a germination rate of ΔMmd::ΔMir was 10%, which was significantly lower than that of ΔMmd and ΔMir (about 50%), while germination rates of ΔMmd and ΔMir were significantly lower than that of WT, C-ΔMmd, and C-ΔMir strains (about 70%) (E in FIG. 7).

In a 1/2SDY liquid medium with 20 mg/L divalent mercury, spores of the mutants ΔMmd::ΔMir, ΔMmd, and ΔMir failed to germinate, but after cultivation for 48 h, germination rates of WT, C-ΔMmd, and C-ΔMir were about 10%, which were increased to about 25% after cultivation for 60 h (F in FIG. 7).

The tolerance of mycelia to methylmercury was further observed. A basic test process was as follows: 100 μL of a spore suspension (10⁷ spores/mL) was uniformly coated on a PDA plate with a diameter of 9 cm and cultivated at 26° C. for 3 d, and a medium-containing mycelium cake was directly collected with a 5 mm puncher, and then inoculated on a PDA plate with methylmercury or divalent mercury and further cultivated, during which a colony diameter was measured every day.

On the ordinary PDA medium, there was no difference in the colony growth among the mutant strains ΔMmd, ΔMir, and ΔMmd::ΔMir, the complementary strains C-ΔMmd and C-ΔMir, and the wild-type strain WT (FIG. 7).

On the PDA medium with 1 mg/L methylmercury, the colony growth of ΔMmd and ΔMmd::ΔMir was significantly inhibited compared with that of WT, while the colony growth of the strains ΔMir, C-ΔMmd, and C-ΔMir showed no difference from that of the WT (FIG. 7).

On the PDA medium with 2 mg/L methylmercury, the ΔMmd and ΔMmd::ΔMir colonies failed to grow, and the colony growth of the strains ΔMir, C-ΔMmd, and C-ΔMir showed no difference from that of the WT (FIG. 7).

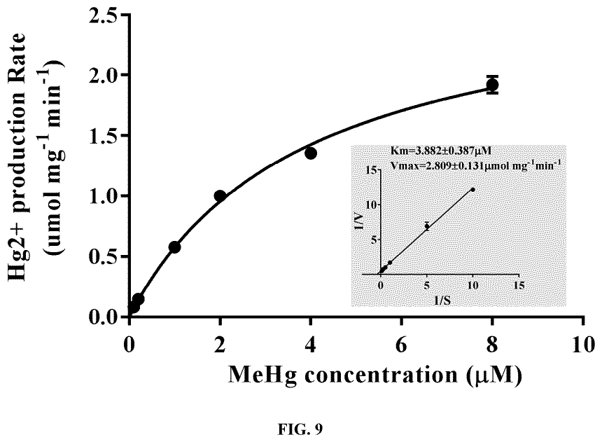

On the PDA medium with 12 mg/L divalent mercury, the colony growth of ΔMmd, ΔMir, and ΔMmd::ΔMir showed no difference from that of the WT (FIG. 8).

On the PDA medium with 16 mg/L divalent mercury, the spore production of ΔMmd, ΔMir, and ΔMmd::ΔMir colonies was reduced to some extent relative to WT, and the colony growth of C-ΔMmd and C-ΔMir showed no difference from that of the WT (FIG. 8).

On the PDA medium with 30 mg/L divalent mercury, the ΔMir and ΔMmd::ΔMir colonies failed to grow; the colony growth of the strains ΔMmd and C-ΔMmd showed no difference from that of the WT; and the C-ΔMir colonies grew faster than WT (FIG. 9).

### 3) Analysis of the Ability of Strains to Remove Methylmercury and Divalent Mercury in the Environment

The abilities of mycelia of the WT strain, the mutants ΔMmd, ΔMir, and ΔMmd::ΔMir, and the complementary strains C-ΔMmd and C-ΔMir to remove methylmercury and divalent mercury in an SDY medium were analyzed. Mycelium preparation and inoculation, and analysis of methylmercury, divalent mercury, and total mercury in a culture supernatant and mycelium were as described above.

After the mycelium (with a wet weight of 0.2 g) was inoculated into 20 mL of an SDY medium with methylmercury (50 μg/L) to treat for 48 h, no methylmercury was detected in a culture supernatant of the WT strain, and only a trace amount of methylmercury was detected in culture supernatants of the strains ΔMir, C-ΔMmd, and C-ΔMir, but a large amount of methylmercury was detected in culture supernatants of the mutants ΔMmd and ΔMmd::ΔMir (still about 30% to 40% of residual methylmercury) (Table 8); no divalent mercury was detected in culture supernatants of the mutants ΔMmd and ΔMmd::ΔMir, and divalent mercury was detected in culture supernatants of the strains WT, ΔMir, C-ΔMmd, and C-ΔMir, where a divalent mercury content in the culture supernatant of the Mir was higher than that of the other three strains; methylmercury was detected in mycelia of all strains, where there was no difference in the methylmercury content in mycelia between the mutants ΔMmd and ΔMmd::ΔMir, there was no significant difference in the methylmercury content among the strains WT, ΔMir, C-ΔMmd, and CA-Mir, and the methylmercury content in mycelia of the former two was significantly higher than that of the latter 4; and there was no divalent mercury in mycelia of ΔMmd and ΔMmd::ΔMir, there was divalent mercury in mycelia of the strains WT, Mir, C-ΔMmd, and C-ΔMir, where ΔMir had the highest divalent mercury content, and there was no significant difference in the divalent mercury among the other three strains (Table 8).

After the mycelium (with a wet weight of 0.2 g) was inoculated into 20 mL of an SDY medium with divalent mercury (10 mg/L) to treat for 48 h, there was no difference in the divalent mercury content among culture supernatants of the strains ΔMir and ΔMmd::ΔMir, but the divalent mercury contents in culture supernatants of the two were significantly higher than that of the strains WT, ΔMmd, C-ΔMmd, and C-ΔMir; the divalent mercury content in a culture supernatant of ΔMmd was also significantly higher than that of WT, C-ΔMmd, and C-ΔMir, and there was no significant difference among the three strains WT, C-ΔMmd, and C-ΔMir; and there was divalent mercury in mycelia of all strains, where mycelia of the two complementary strains C-ΔMmd and C-ΔMir had a lower divalent mercury content than other strains, and there was no significant difference among the other strains (Table 9).

TABLE 8
Methylmercury and total mercury contents in the SDY supernatant and mycelium

50 μg/L of

| methylmercury was | | | | | | | Total |
|---|---|---|---|---|---|---|---|
| added to 20 mL of | Methylmercury content (ng) | | | Divalent mercury content (ng) | | | mercury |
| SDY (Total: 1,000 ng) | Supernatant | Mycelium | Sum | Supernatant | Mycelium | Sum | (ng) |
| WT | ND | 5 ± 1.5^{bc} | 5 ± 1.5^{d} | 108.4 ± 14.5^{b} | 7.47 ± 1.33^{b} | 115.9 ± 15.7^{b} | 795 ± 30^{a} |
| ΔMmd | 251.9 ± 41.6^{b} | 31.3 ± 9.2^{a} | 283.2 ± 50.6^{b} | ND | ND | ND | 781.3 ± 21.8^{a} |
| C-ΔMmd | 3.5 ± 1.1^{c} | 4.0 ± 0.6^{c} | 7.5 ± 1.0^{cd} | 91.9 ± 14.7^{b} | 6.47 ± 1.25^{b} | 98.4 ± 15.96^{b} | 793.3 ± 99.4^{a} |
| ΔMir | 2.3 ± 0.5^{c} | 8.3 ± 0.6^{b} | 12.6 ± 1.8^{c} | 163.7 ± 22.1^{a} | 10.97 ± 1.66^{a} | 174.7 ± 22.4^{a} | 800 ± 28.9^{a} |
| C-ΔMir | 4.4 ± 0.9^{c} | 6.8 ± 1.2^{bc} | 11.2 ± 1.3^{c} | 87.4 ± 11.4^{b} | 5.77 ± 1.68^{b} | 93.2 ± 13.0^{b} | 861.4 ± 73.9^{a} |
| ΔMmd::ΔMir | 277.5 ± 52.4^{b} | 20 ± 5.8^{a} | 297.5 ± 57.8^{b} | ND | ND | ND | 799.7 ± 45.4^{a} |
| Untreated control | 753.3 ± 29.1^{a} | — | 753.3 ± 29.1^{a} | ND | — | ND | 827.7 ± 29.9^{a} |

TABLE 9
Divalent mercury and total mercury contents in the SDY supernatant and mycelium

| 10 mg/L of methylmercury | | | | |
|---|---|---|---|---|
| is added to 20 mL of | Divalent mercury content (μg) | | | Total mercury |
| SDY (Total: 200 μg) | Supernatant | Mycelium | Sum | (μg) |
| WT | 73.3 ± 1.5^{d} | 42.9 ± 1.5^{b} | 116.2 ± 1.8^{d} | 172.3 ± 6.1^{a} |
| ΔMmd | 82.6 ± 0.5^{c} | 48.5 ± 1.5^{a} | 131.1 ± 1.7^{c} | 169.9 ± 3.3^{a} |
| C-ΔMmd | 76.1 ± 0.4^{d} | 34.5 ± 0.6^{c} | 110.5 ± 0.7^{d} | 165.6 ± 2.9^{a} |
| ΔMir | 90.3 ± 0.5^{b} | 46.1 ± 3.3^{ab} | 136.4 ± 3.8^{bc} | 159.8 ± 1.6^{a} |
| C-ΔMir | 75.6 ± 0.8^{d} | 34.0 ± 4.2^{c} | 109.7 ± 3.6^{d} | 167.3 ± 4.4^{a} |
| ΔMmd::ΔMir | 92.3 ± 0.5^{b} | 52.6 ± 3.8^{a} | 144.9 ± 3.8^{ab} | 163.7 ± 1.2^{a} |
| Untreated control | 159.3 ± 7.7^{a} | — | 159.3 ± 7.7^{a} | 173.7 ± 5.0^{a} |

### Example 6 Expression, Purification and Activity Analysis of the MMD Protein

1) Expression and Purification of the MMD Protein in the E. coli Strain BL21

A prokaryotic expression vector of MMD was constructed as follows: (1) A coding sequence of MMD was amplified by PCR, and primers used were shown in Table 1. (2) An amplification product and a vector pET-28a-sumo were each digested with EcoR I and BamH I, and digestion products were recovered, ligated, and transformed into the E. coli strain DH5αu. Positive clones were verified by sequencing to obtain a vector pET-28a-sumo-MMD. (3) DNA for the vector pET-28a-sumo-MMD was prepared and transformed into the E. coli strain BL21 for prokaryotic expression.

The prokaryotic expression was conducted as follows: the E. coli strain BL21 carrying the vector pET-28a-sumo-MMD was inoculated into an LB liquid medium (with kanamycin), and then cultivated at 37° C. under shaking at 220 rpm until OD₆₀₀ of a resulting bacterial solution was 0.6 to 1.0; and then isopropyl-o-D-thiogalactoside (IPTG) (0.8 mM) was added, and the strain was further cultivated at 18° C. for 12 h to 16 h to induce the expression of MMD.

The protein was purified as follows: (1) After the induced protein expression was completed, bacterial cells were collected by centrifugation at 4° C. and 4,500 rpm for 25 min, then resuspended in a lysis buffer with a pH of 7.0, and then subjected to ultrasonication (70 kHz, 25 min). A resulting mixture was centrifuged at 4° C. and 12,000 rpm for 50 min to obtain a supernatant, and the nickel column affinity chromatography was used to preliminarily separate and purify the fusion protein SUMO::MMD. HispurTMNi-NTA Resin was used as a packing of the chromatography column. Impurities were rinsed away using a column washing liquid (pH 7.0), and then an elution buffer (pH 7.0) was used to elute the fusion protein SUMO::MMD on the column. (2) The protease ULP1 was used to cut off the SUMO tag on the fusion protein SUMO::MMD. (3) ULP and SUMO proteins were separated from the MMD protein by the nickel column affinity chromatography to obtain pure MMD protein. (3) The Amino Ultra-15 (10 kDa) ultrafiltration tube was used to concentrate the MMD pure protein obtained above and remove the imidazole left in the solution during the protein purification process. Glycerol was added to a resulting protein solution to a final concentration of 10%, and then the protein solution was stored at −80° C.

### 2) Detection of Methylmercury Demethylation Activity of MMD

(1) Activity determination Reaction system in a total volume of 200 μL: 50 mM sodium phosphate buffer (pH 7.4), 5 mM ethylenediaminetetraacetic acid (EDTA), 0.2 mM magnesium acetate, 0.5 mM L-cysteine, 0.5 mg/mL bovine serum albumin (BSA), concentration-gradient methylmercury (0.5 μM, 1 μM, 2 μM, 4 μM. and 8 μM), and 5 μg MMD protein. The reaction system was incubated at 37° C. for 1 h, and then methylmercury and divalent mercury contents in the reaction system were detected by HPLC-ICP-MS.

Results: The production of divalent mercury in the enzymatic reaction system was detected by HPLC-ICP-MS, confirming that the MMD has the activity of degrading methylmercury. The results were shown in FIG. 9.

(2) Vₘₐₓ and Kₘ analysis in order to detect the Vₘₐₓ and Kₘ values of the MMD enzyme, in the above reaction system, except for the change in protein and methylmercury, other conditions remained unchanged. The setting of methylmercury and protein concentrations was shown in Table 2. Results were shown in FIG. 9.

Although the present disclosure has been described in detail through the above examples, the examples are only a part rather than all of the examples of the present disclosure. All other examples obtained by persons based on these examples without creative efforts shall fall within a protection scope of the present disclosure.

1. A fungus for treating mercury contamination, wherein the fungus expresses a methylmercury demethylase (MMD) and a mercury ion reductase (MIR); and the fungus comprises a Metarhizium fungus and a non-Metarhizium fungus, and the non-Metarhizium fungus comprises Fusarium oxysporum, Oidiodendron maius, Pyronema omphalodes, Amorphotheca resinae, Cadophora malorum, Hyaloscypha bicolor, Pseudogymnoascus sp, and Exophiala oligosperma.

2. The fungus according to claim 1, wherein a gene encoding the MMD comprises a homologous gene with a Genbank accession number of XP_007825874 or XP_007825874; and a gene encoding the MIR comprises a homologous gene with a Genbank accession number of XP_007824121 or XP_007824121.

3. The fungus according to claim 1, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

4. A microbial agent for methylmercury demethylation and divalent mercury reduction, comprising at least one of the fungi according to claim 1.

5-11. (canceled)

12. A method for removing methylmercury and divalent mercury in soil, comprising the following steps: planting a plant that has a symbiotic relationship with the fungus according to claim 1 in the soil, and inoculating the fungus.

13. The method according to claim 12, wherein the plant that has a symbiotic relationship with the fungus comprises an herbaceous plant and a woody plant, and the herbaceous plant comprises a gramineous plant.

14. The method according to claim 13, wherein the gramineous plant comprises Pennisetum purpureum (P. purpureum) and/or Zea mays L., and the woody plant comprises Morus alba L. and/or Acer spp.

15. The method according to claim 12, wherein the inoculation comprises: irrigating roots with a spore suspension of the fungus; and roots of each plant are irrigated with 10 mL of the spore suspension, and each mL of the spore suspension comprises 1×105 spores.

16. (canceled)

17. The fungus according to claim 2, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

18. The microbial agent according to claim 4, wherein a gene encoding the MMD comprises a homologous gene with a Genbank accession number of XP_007825874 or XP_007825874; and a gene encoding the MIR comprises a homologous gene with a Genbank accession number of XP_007824121 or XP_007824121.

19. The microbial agent according to claim 4, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

20. The microbial agent according to claim 5, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

21. The method according to claim 12, wherein a gene encoding the MMD comprises a homologous gene with a Genbank accession number of XP_007825874 or XP_007825874; and a gene encoding the MIR comprises a homologous gene with a Genbank accession number of XP_007824121 or XP_007824121.

22. The method according to claim 12, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

23. The method according to claim 21, wherein the Metarhizium fungus comprises Metarhizium robertsii (M. robertsii), Metarhizium anisopliae (M. anisopliae), Metarhizium brunneum (M. brunneum), Metarhizium guizhouense (M. guizhouense), Metarhizium majus (M. majus), and Metarhizium acridum (M. acridum);
the M. robertsii has an accession number of USDA ARSEF2575, the M. anisopliae has an accession number of USDA ARSEF549, the M. brunneum has an accession number of USDA ARSEF3297, the M. guizhouense has an accession number of USDA ARSEF977, the M. majus has an accession number of USDA ARSEF297, and the M. acridum has an accession number of USDA ARSEF324; and
the Fusarium oxysporum has an accession number of NRRL 32931, the Cadophora malorum has an accession number of bio-12245, the Oidiodendron maius has an accession number of ATCC 60377, the Hyaloscypha bicolor has an accession number of CBS144009, the Pseudogymnoascus sp. has an accession number of ATCC MYA-4855, the Pyronema omphalodes has an accession number of ATCC 14881, the Exophiala oligosperma has an accession number of ATCC28180, and the Amorphotheca resinae has an accession number of ATCC 22711.

24. The method according to claim 21, wherein the plant that has a symbiotic relationship with the fungus comprises an herbaceous plant and a woody plant, and the herbaceous plant comprises a gramineous plant.

25. The method according to claim 22, wherein the plant that has a symbiotic relationship with the fungus comprises an herbaceous plant and a woody plant, and the herbaceous plant comprises a gramineous plant.

26. The method according to claim 23, wherein the plant that has a symbiotic relationship with the fungus comprises an herbaceous plant and a woody plant, and the herbaceous plant comprises a gramineous plant.

27. The method according to claim 24, wherein the gramineous plant comprises Pennisetum purpureum (P. purpureum) and/or Zea mays L., and the woody plant comprises Morus alba L. and/or Acer spp.

28. The method according to claim 25, wherein the gramineous plant comprises Pennisetum purpureum (P. purpureum) and/or Zea mays L., and the woody plant comprises Morus alba L. and/or Acer spp.


SEQUENCE LISTING (28 sequences)

SEQ ID NO: 1 - DNA, 42 bp, Artificial Sequence
  DMmd-5-1
ggggacagctttcttgtacaaagtggatttagcaaacgacca

SEQ ID NO: 2 - DNA, 42 bp, Artificial Sequence
  DMmd-5-2
ggggactgcttttttgtacaaacttgtcagactcaacagcca

SEQ ID NO: 3 - DNA, 42 bp, Artificial Sequence
  DMmd-3-1
ggggacaactttgtatagaaaagttgttttggtatgacctgc

SEQ ID NO: 4 - DNA, 42 bp, Artificial Sequence
  DMmd-3-2
ggggacaactttgtataataaagttgtgagacttggaatcgt

SEQ ID NO: 5 - DNA, 18 bp, Artificial Sequence
  DMmd-CF1
aggactggtgttggagag

SEQ ID NO: 6 - DNA, 17 bp, Artificial Sequence
  DMmd-CF2
caggtcatcggatgaag

SEQ ID NO: 7 - DNA, 25 bp, Artificial Sequence
  gMmd-5
gggctagcctccaagacagtacgtg

SEQ ID NO: 8 - DNA, 25 bp, Artificial Sequence
  gMmd-3
gggatatcgagcaactgtgtacttg

SEQ ID NO: 9 - DNA, 18 bp, Artificial Sequence
  cc-Mmd-5
aagattcaactgctgacc

SEQ ID NO: 10 - DNA, 18 bp, Artificial Sequence
  cc-Mmd-3
acaaagttgacgaccaag

SEQ ID NO: 11 - DNA, 25 bp, Artificial Sequence
  DMir-5-1
ggtctagactgtttggcagcatcat

SEQ ID NO: 12 - DNA, 25 bp, Artificial Sequence
  DMir-5-2
gggaattcttgacatggcctatcac

SEQ ID NO: 13 - DNA, 25 bp, Artificial Sequence
  DMir-3-1
ggtttaaatggctgaatgtagccgt

SEQ ID NO: 14 - DNA, 25 bp, Artificial Sequence
  DMir-3-2
ggtttaaaccttcgaagctggccat

SEQ ID NO: 15 - DNA, 17 bp, Artificial Sequence
  DMir-CF1
gacaatatggctgaatg

SEQ ID NO: 16 - DNA, 17 bp, Artificial Sequence
  DMir-CF2
cgatctccgttatggtc

SEQ ID NO: 17 - DNA, 25 bp, Artificial Sequence
  gMir-5
ggactagtgcataactaaggaagtt

SEQ ID NO: 18 - DNA, 25 bp, Artificial Sequence
  gMir-3
gggatatcctatttcaacgagcccc

SEQ ID NO: 19 - DNA, 17 bp, Artificial Sequence
  cc-Mir-5
ttatcaccgataaagga

SEQ ID NO: 20 - DNA, 17 bp, Artificial Sequence
  cc-Mir-3
atcccagaatcttctgc

SEQ ID NO: 21 - DNA, 25 bp, Artificial Sequence
  E-Mmd-5
ggggatccatgagtcaacagagccc

SEQ ID NO: 22 - DNA, 25 bp, Artificial Sequence
  E-Mmd-3
gggaattctcaagacaacccgtacc

SEQ ID NO: 23 - DNA, 18 bp, Artificial Sequence
  Bar-up
cgcctggacgactaaacc

SEQ ID NO: 24 - DNA, 18 bp, Artificial Sequence
  Bar-down
tcagcctgccggtaccgc

SEQ ID NO: 25 - DNA, 18 bp, Artificial Sequence
  Sur-up
atcgtggagtcatgtttg

SEQ ID NO: 26 - DNA, 18 bp, Artificial Sequence
  Sur-down
ccagtaagtaatatatcc

SEQ ID NO: 27 - DNA, 19 bp, Artificial Sequence
  NTC-up
catccactgcacctcagag

SEQ ID NO: 28 - DNA, 19 bp, Artificial Sequence
  NTC-down
gtaccggcggatggggttc